(19) United States
(12) Patent Application Publication
(10) Pub. No.: US 2012/0102591 A1
(43) Pub. Date: Apr. 26, 2012

(54) Enhancing Salt Tolerance of Plants with Rice OsNHAD Gene

(75) Inventors: Lizhong Xiong, Hubei Province (CN); Xin Hou, Hubei Province (CN); Zhuyun Qi, Hubei Province (CN)

(73) Assignee: HUAZHONG AGRICULTURAL UNIVERSITY, Wuhan (CN)

(21) Appl. No.: 13/264,620

(22) PCT Filed: Apr. 14, 2010
(86) PCT No.: PCT/CN2010/000490

§ 371 Date: Jan. 16, 2012

(30) Foreign Application Priority Data
Apr. 16, 2009 (CN) 200910061607.X

(51) Int. Cl. C12N 15/82

(52) U.S. Cl. 800/278

(57) ABSTRACT

The present invention pertains to the field of rice genetic engineering. Specifically, the present invention relates to a rice OsNHAD gene that enhances tolerance to salt stress, which was obtained through gene isolation, cloning and function verification, and also to use of the gene in genetic improvement of salt tolerance of rice. Said gene is selected from one of the following nucleotide sequences: 1) the nucleotide sequence from positions 60 to 1649 of SEQ NO: 1 in the Sequence Listing; or 2) a nucleotide sequence that encodes the same protein as that encoded by 1). Transgenic rice plants obtained by introducing into rice the nucleotide sequence comprising OsNHAD gene operably ligated with exogenous promoter had enhanced salt tolerance.

### TECHNICAL FIELD

The present disclosure pertains to the field of rice genetic engineering. Specifically, the present disclosure relates to a rice OsNHAD gene that enhances tolerance to salt stress, which was obtained through gene isolation, cloning and function verification, and also to use of the gene in genetic improvement of salt tolerance of rice. OsNHAD gene is associated with tolerance of plants to non-biological stresses. Transgenic rice plants obtained by introducing into rice the complete coding sequence of the gene ligated with cauliflower mosaic virus promoter (CaMV35S) had enhanced tolerance to high salt stress.

### BACKGROUND ART

Although the growth of a plant is dictated by the plant's inherent genetic makeup, it is usually susceptible to a wide variety of environmental factors. Drought, high salt and low temperature are the most common non-biological stresses that severely influence the growth and limit the distribution of plants. Non-biological stresses will result in decline in yields and quality of crops, representing a bottleneck for agricultural development in many regions. Therefore, it has always been one of the main objects of agricultural science and technology research to breed stress-resistant crop varieties. In order to adapt to or resist against these stress conditions, plants have, through prolonged acclimatization, developed a set of self-protection mechanisms to protect them from stresses such as drought, high salt and low temperature. Drought, high salt and low temperature stresses may disrupt ionic balance in plant cells and dehydrate the cells, such that the cells are subject to ionic and water stresses, resulting in changes in gene expression, metabolism and morphology of the plant, which includes, among others, increased or decreased expression of some genes, retarded or oven ceased growth, transient rise in hormones (such as ABA), and aggregation of substances for regulating osmotic pressure (Seki M, Umezawa T, Urano K, Shinozaki K. Regulatory metabolic networks in drought stress responses. Curr Opin Plant Biol, 2007, 10: 296-302). When a plant is subject to stresses, a series of signal transduction and transcription regulation are initiated to express a variety of downstream stress-resistant genes. The products encoded by these downstream stress-resistant genes are mainly proteins that play a direct protective role in tolerance of the plant to stresses, including functional proteins that protect cells from damage by water stress, key enzymes for synthesizing osmosis-regulating substances, and enzymes for eliminating reactive oxygen species (ROS), etc. These proteins can increase tolerance of the plant to stresses, such as chaperone proteins, LEA proteins, antifreeze proteins, channel proteins, antioxidant proteins, etc (Valliyodan B, Nguyen H T. Understanding regulatory networks and engineering for enhanced drought tolerance in plants. Curr Opin Plant Biol, 2006, 9: 189-195). These functional proteins play a very important role in the process of response of the plant to stresses. Therefore, it is of great significance to isolate and identify those stress-resistant functional genes and apply them to genetic improvement of crops against stresses. Attempts have been made to improve stress tolerance of plants based on studies of existing model plants. For example, LEA proteins are highly hydrophilic such that the plant can be protected from damage to cellular membrane systems and biomacromolecules when there is a lack of water. Transgenic rice plants introduced with barley LEA protein gene HVA1 showed markedly increased tolerance to drought and salt in comparison to control plants, with the strength of tolerance being in clear correlation with the content of LEA proteins (Xu D, Duan X, Wang B, Hong B, Ho T, Wu R. Expression of a Late Embryogenesis Abundant Protein Gene, HVA1, from Barley Confers Tolerance to Water Deficit and Salt Stress in Transgenic Rice. Plant Physiol, 1996, 110: 249-257). Na⁺/H⁺ antiporter gene AtNHX1 of Arabidopsis thaliana can not only increase salt tolerance of this plant when expressed (Shi H, Zhu J K. Regulation of expression of the vacuolar Na⁺/H⁺ antiporter gene AtNHX1 by salt stress and abscisic acid. Plant Mol Biol, 2002, 50: 543-550), but also markedly enhance salt tolerance of transgenic tomato plants into which it is introduced (Zhang H X, Blumwald E. Transgenic salt-tolerant tomato plants accumulate salt in foliage but not in fruit. Nat Biotechnol, 2001, 19: 765-768).

Rice (Oryza sativa) is one of the most important grain crops and therefore it is of great significance to breed novel stress-resistant rice varieties. In our earlier studies, a cDNA was isolated which is subject to induction by a variety of stresses. Sequence analysis of it revealed a certain degree of similarity to bacterial NHAD protein. Bacterial NHAD protein enhances tolerance of transgenic bacteria to high salt stress through transport of Na⁺/H⁺ ions (Habibian R, Dzioba J, Barrett J, Galperin M Y, Loewen P C, Dibrov P. Functional analysis of conserved polar residues in Vc-NhaD, Na⁺/H⁺ antiporter of Vibrio cholerae. J Biol Chem, 2005, 280: 39637-39643). However, there is still no report as to whether overexpression of NHAD protein in plants can increase stress tolerance of the plants. Therefore, isolating a homologous gene to NHAD from rice and identifying the role it plays in increasing stress tolerance of rice would be of great significance in breeding novel stress-resistant rice varieties.

### SUMMARY OF THE INVENTION

One object of the present invention is to isolate and clone from rice a DNA segment (in the present application, “DNA segment” is synonymous with “nucleotide sequence”, and the same applies below) comprising the complete coding region of the homologous gene for the functional protein, use the gene to increase tolerance of rice to salt stress, and use of the gene in genetic improvement of salt tolerance of rice. Analysis of the protein sequence encoded by the gene revealed that the protein has some degree of similarity to bacterial NHAD (Na⁺/H⁺ antiporter D type) protein, therefore the gene was designated as OsNHAD gene.

The present disclosure relates to isolation and use of a DNA segment comprising OsNHAD gene, which confers plants with enhanced tolerance to stresses. OsNHAD gene is selected from one of the following nucleotide sequences:

1) the DNA sequence from positions 60 to 1649 of SEQ NO: 1 in the Sequence Listing; or

2) a DNA sequence that encodes the same protein as that encoded by 1).

The gene of the present invention or a homologous gene thereof can be obtained by screening a cDNA library or genomic library using a cloned OsNHAD gene as the probe. Alternatively, OsNHAD gene according to the present invention and any DNA segments of interest or homologous DNA segments thereof can be obtained by amplification from genome, mRNA and cDNA using PCR (polymerase chain reaction) technology. The sequence containing OsNHAD gene can be isolated using the above methods. By transforming plants with said isolated sequence incorporated in any expression vector that can direct the expression of an exogenous gene in plant, transgenic plants with enhanced tolerance to stresses can be produced. In the process of constructing the gene according to the present invention into plant expression vector, any strong promoter or inducible promoter can be inserted into the position preceding the transcription initiation nucleotide, or alternatively, an enhancer may be used. Such an enhancer region can be ATG initiation codon and initiation codon in flanking regions and the like, provided that the enhancer region is in frame with the coding sequence to ensure the translation of a complete sequence.

The expression vector bearing OsNHAD gene according to the present invention can be introduced into plant cells by conventional biotechnological methods such as Ti plasmid, plant viral vector, direct DNA transformation, microinjection, electroporation and the like (Weissbach, 1998, Method for Plant Molecular Biology VIII, Academy Press, New York, pp. 411-463; Geiserson and Corey, 1998, Plant Molecular Biology (2^{nd} Edition)).

The expression vector comprising OsNHAD gene according to the present invention can be used to transform a host which is selected from a wide variety of plants including rice, so as to breed drought-, salt- and cold-tolerant plant varieties.

As the gene according to the present invention is induced by stresses, it can be ligated with any stress-inducible promoter of interest and introduced into a suitable expression vector which is then transformed into a host plant. The transgenic plants obtained may be induced to express the gene under stress conditions, resulting in their increased tolerance to non-biological stresses.

The invention will now be described more fully with reference to drawings and examples.

### BRIEF DESCRIPTION OF THE DRAWINGS

SEQ ID No: 1 in the Sequence Listing shows the nucleotide sequence isolated and cloned according to the present invention, which comprises the coding region of OsNHAD gene.

FIG. 1 shows the result of alignment of the predicted protein sequence of OsNHAD gene with homologous NHAD protein sequence using ClustalW software (a publicly used software), wherein:

Os: gi 115477946, Sequence source: Oryza sativa (rice);

Zm: gi 195611882, Sequence source: Zea mays (corn);

At1: gi 18402254, Sequence source: Arabidopsis thaliana;

At2: gi 15222822, Sequence source: Arabidopsis thaliana;

Mc: gi 150247011, Sequence source: Mesembryanthemum crystallinum (ice plant);

Bj: gi 27378850, Sequence source: Bradyrhizobium japonicum (soybean Rhizobium).

FIG. 2 schematically shows the construction of overexpression vector pCB2004H-OsNHAD according to the present invention. The full-length OsNHAD gene was inserted behind CaMV35S promoter via recombination reaction.

FIG. 3 shows the expression levels of OsNHAD gene in various tissues of rice detected by real-time PCR. The ten tissues or organs are: 1) callus; 2) seed; 3) three-day-old shoot; 4) leaf and root at trefoil stage; 5) flag leaf; 6) stem; 7) young spike shorter than 5 cm; 8) extruded spike; 9) glume; and 10) endosperm. For all tissues/organs except callus, the expression level of OsNHAD gene was referenced to that in callus (assumed to be 1) to obtain the relative expression level.

FIG. 4 (including 4a, 4b, 4c and 4d) shows changes in expression level of OsNHAD gene following stress treatment (drought, high salt, low temperature, abscisic acid (ABA)), as detected by real-time PCR. The expression levels of OsNHAD gene after treatment were referenced to that before treatment (assumed to be 1).

FIG. 5 shows a comparison of the growth of five transgenic rice lines (T₁) overexpressing OsNHAD and control line, grown under normal condition and 200 mmol/L high salt stress.

### EXAMPLES

The following examples illustrate the present invention, describing the methods for isolating and cloning the DNA segment comprising the complete coding region of OsNHAD gene, as well as the method of verifying the function of OsNHAD gene. In light of the following description and these examples, the basic features of the present invention will be acknowledged by one skilled artisan, and various changes and modifications to the present invention can be made to adapt to various uses and conditions without departing from the spirit and scope of the present invention.

### Example 1

### Isolation and Cloning of OsNHAD Gene

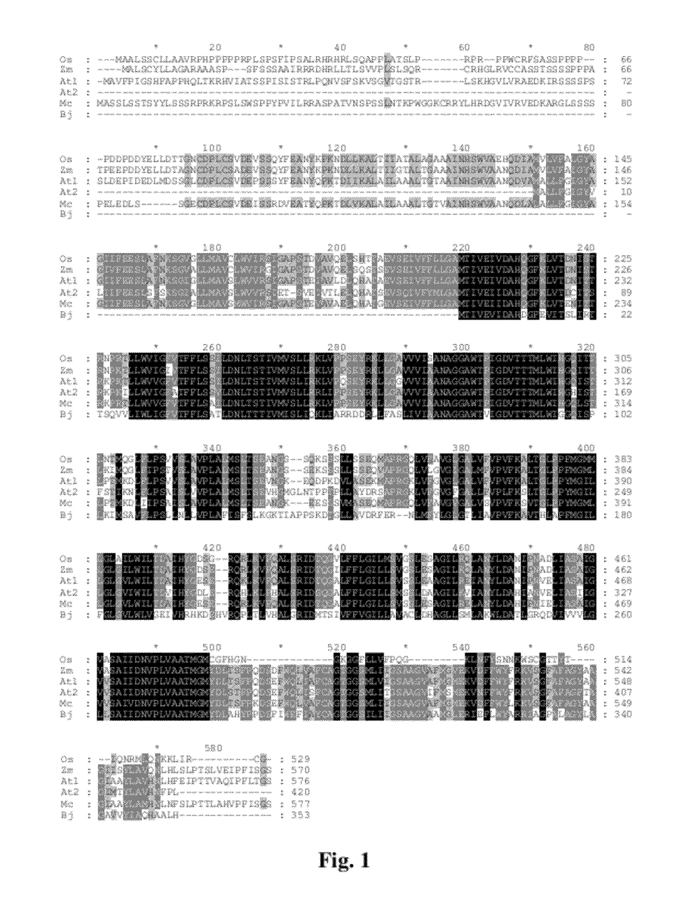

Through analysis of the expression profiles of drought inducible genes of the rice variety “Zhonghan 5” (a publicly used rice variety available from Shanghai Academy of Agricultural Sciences, China) and “Zhenshan 97” (a widely used parent for hybrid rice production in China), an EST (expression sequence tag) whose expression was upregulated when induced by drought was found. Sequence analysis on the product encoded by this OsNHAD gene indicated it had 71% homology to NHAD (a protein from soybean Rhizobium) (FIG. 1). The gene's corresponding cDNA clone J013023H14 was found by searching Knowledged-based Oryza Molecular Biological Encyclopedia of Japan (http://cdna( )1.dna.affrc.go.jp), and mapped to 110206 bp to 122237 bp of BAC clone AP005787 on chromosome 9. Total RNA was extracted from leaves of the drought-treated rice variety “Nipponbare” (a published rice variety) using TRIZOL reagent (Invitrogen Corp., performed according to the manufacturer's instruction), and then reverse transcribed into cDNA with reverse transcriptase SSII (purchased from Invitrogen Corp.) under the following conditions: 65° C. for 5 min, 42° C. for 120 min, and 70° C. for 10 min The full-length cDNA of OsNHAD gene was amplified using primers GPF (5′-GGCACTCTCACTCACACGG-3′) and GPR (5′-GGATGGTCCCAATGTAACCC-3′). PCR reaction was performed under the following conditions: pre-denaturing at 94° C. for 3 min; 35 cycles of 94° C. for 30 sec, 53° C. for 30 sec, and 72° C. for 4 min; and extension at 72° C. for 10 min. The amplified PCR products were ligated into pGEM-T vector (purchased from Promega Co., Ltd). The desired full-length cDNA of the gene was obtained by screening for a positive clone and sequencing. The positive clone obtained was designated as pGEM-OsNHAD.

### Example 2

### Construction and Genetic Transformation of OsNHAD Gene Overexpression Vector

In order to better analyze the function of OsNHAD gene, it was overexpressed in rice for studying its function by observing the phenotypes of transgenic plants.

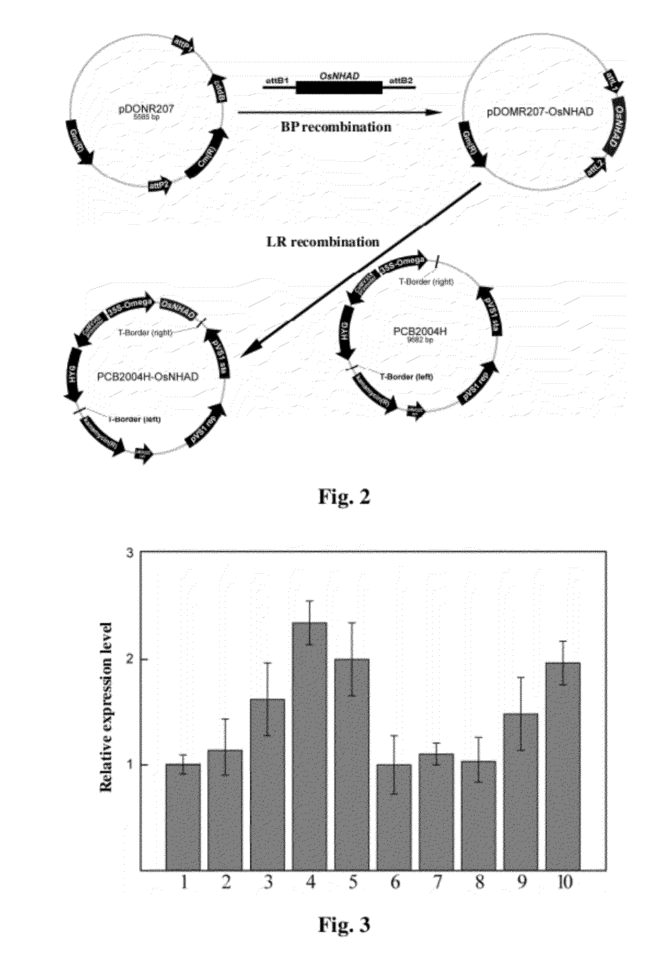

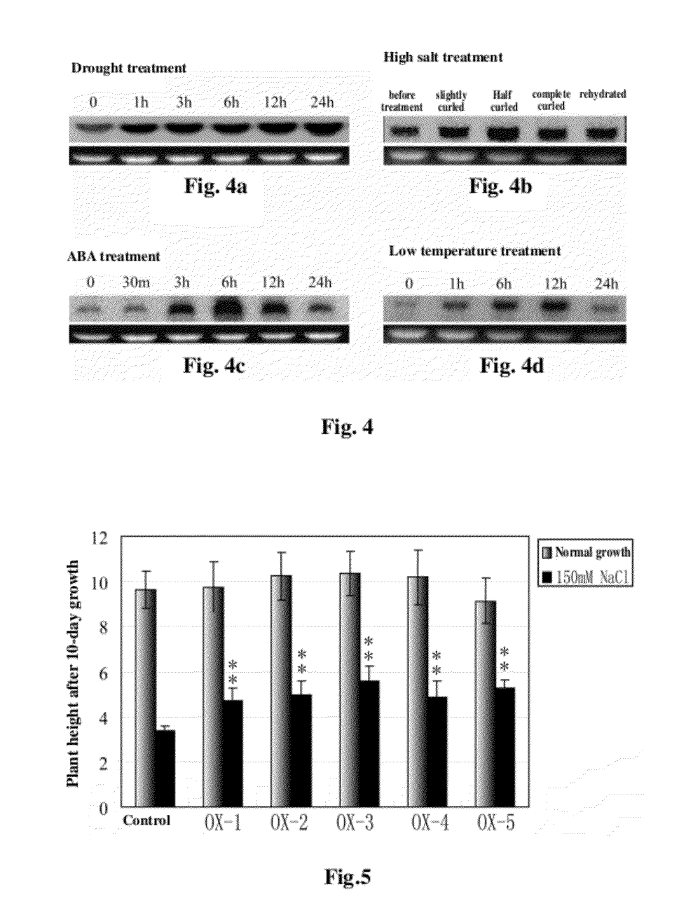

The overexpression vector was constructed as follows. Firstly, the positive clone pGEM-OsNHAD plasmid obtained in Example 1 was amplified using primers ALLF (5′-GGG GAC AAG TTT GTA CAA AAA AGC AGG CTT AAT ACG ACT CAC TAT AGG G-3′) and ALLR (5′-GGG GAC CAC TTT GTA CAA GAA AGC TGG GTA TTT AGG TGA CAC TAT AG-3′) to obtain a DNA segment comprising full-length OsNHAD gene. The reaction conditions were: pre-denaturing at 94° C. for 3 min; 35 cycles of 94° C. for 30 sec, 50° C. for 30 sec, and 72° C. for 4 min; and extension at 72° C. for 10 min The DNA segment was constructed into pDONR207 vector (purchased from Invitrogen Corp.) via BP recombination reaction according to the manufacturer's instructions of Invitrogen recombination cloning kit. Then LR recombination was performed on pDONR207-OsNHAD and pCB2004H vector (a public available overexpression vector, Lei Z, et al. (2007) High-throughput binary vectors for plant gene function analysis. J Int Plant Biol 49:556-567) according to the manufacturer's instructions of Invitrogen recombination cloning kit (Cat. No. 11791-020). Thus OsNHAD gene was finally constructed into the overexpression vector pCB2004H useful for genetic transformation (see FIG. 2).

The overexpression vector pCB2004H was introduced into the rice variety “Zhonghua 11” (a publicly used rice variety, available from China National Rice Research Institute) using rice genetic transformation system mediated by Agrobacterium. Transgenic plants were obtained through pre-cultivation, infection, co-cultivation, screening the calli with hygromycin resistance, differentiation, rooting, hardening of seedling and transplantation. The rice (japonica rice subspecies) genetic transformation system mediated by Agrobacterium was modified on the basis of the method reported by Hiei, et al. (Hiei, et al., Efficient transformation of rice, Oryza sativa L., mediated by Agrobacterium and sequence analysis of the boundaries of the T-DNA, Plant Journal 6:271-282, 1994). A total of 40 independent transgenic rice plants were obtained using the transformation vector.

The procedure was carried out as follows: (1) Callus Induction: Mature rice seeds were husked, and then were successively treated with 70% alcohol for 1 minute and surface-disinfected with 0.15% HgCl₂ for 15 minutes. The seeds were rinsed with sterilized water for 4-5 times. The treated seeds were put onto the induction medium (the formulation thereof is as described below). The seeded medium was placed in darkness for 4-week culture at 25±1° C. (2) Subculture: Bright yellow, compact and relatively dry embryogenic calli were selected, put onto subculture medium as described below, and cultured in darkness for 2 weeks at 25±1° C. (3) Pre-culture: The compact and relatively dry embryogenic calli were selected, put onto the pre-culture medium as described below, and cultured in darkness for 2 weeks at 25±1° C. (4) Agrobacterium Culture: Agrobacterium EHA105 (a commercial strain, available from CAMBIA) was pre-cultured on the LA medium with corresponding resistance selection at 28° C. for 2 days. Then the Agrobacterium was transferred to the suspension medium as described below and cultured on a shaker at 28° C. for 2-3 hours. (5) Agrobacterium Infection: The pre-cultured calli were transferred into a sterilized glass bottle. The Agrobacterium suspension was adjusted to OD₆₀₀ 0.8-1.0. The calli were immersed in the Agrobacterium suspension for 30 minutes and then transferred onto a sterilized filter paper and dried. The dried calli were put onto the co-culture medium as described below for 3 days at 19-20° C. (6) Washing and Selective Culture of Calli: The calli were washed with sterilized water until no Agrobacterium was observed. The washed calli were immersed in sterilized water containing 400 ppm carbenicillin (CN) for 30 minutes and then transferred onto a sterilized filter paper and dried. The dried calli were transferred onto the selective medium as described below and screened for 2-3 times, 2 weeks for each time (the concentration of carbenicillin was 400 ppm for the first round screening and 250 ppm for later rounds screenings, and the concentration of hygromycin was 250 ppm). (7) Differentiation: The resistant calli obtained were transferred to the pre-differentiation medium as described below, and cultured in darkness for 5-7 weeks. The pre-differentiated calli were transferred to the differentiation medium as described below, and cultured under light at 26° C. (8) Rooting: The roots of the plantlets generated during differentiation were cut off. Then the plantlets were transferred to the rooting medium as described below, and cultured under light at 26° C. for 2-3 weeks. (9) Transplantation: The residual medium on the roots of the plantlets was washed off, and those plantlets with good root system were transferred into a greenhouse. The greenhouse was maintained moisturized in the first few days of transplantation.

### Formulation of the Reagents

(1) Abbreviations for Reagents and Solutions: The abbreviations for phytohormones used in culture media of the present invention are as follows: 6-BA (6-Benzylaminopurine); CN (Carbenicillin); KT (Kinetin); NAA (Naphthaleneacetic acid); IAA (Indole-3-acetic acid); 2,4-D (2,4-Dichlorophenoxyacetic acid); AS (Acetosyringone); CH (Casein Hydrolysate); HN (Hygromycin); DMSO (Dimethyl Sulfoxide); N6max (macroelement solution for N6 basal medium); N6mix (microelement solution for N6 basal medium); MSmac (macroelement solution for MS basal medium); and MSmic (microelement solution for MS basal medium).

### (2) Formulae of Primary Solutions

1) Preparation of macroelement mother solution for N6 basal medium (10× concentrate):

| | |
|---|---|
| Potassium nitrate (KNO₃) | 28.3 g |
| Potassium dihydrogen phosphate | 4.0 g |
| (KH₂PO₄) | |
| Ammonium sulfate ((NH₄)₂SO₄) | 4.63 g |
| Magnesium sulplate (MgSO₄ • 7H₂O) | 1.85 g |
| Potassium chloride (CaCl₂ • 2H₂O) | 1.66 g |

These compounds were dissolved in succession with distilled water and then the volume was brought to 1000 ml with distilled water at room temperature.

2) Preparation of microelement mother solution for N6 basal medium (100× concentrate):

| | |
|---|---|
| Potassium iodide (KI) | 0.08 g |
| Boric acid (H₃BO₃) | 0.16 g |
| Manganese sulfate (MnSO₄ • 4H₂O) | 0.44 g |
| Zinc sulfate (ZnSO₄ • 7H₂O) | 0.15 g |

These compounds were dissolved in distilled water and then the volume was brought to 1000 ml with distilled water at room temperature.

3) Preparation of iron salt (Fe₂EDTA) stock solution (100× concentrate):

800 ml double distilled water was prepared and heated to 70° C., then 3.73 g Na₂EDTA.2H₂O was added and fully dissolved. The resulting solution was kept in 70° C. water bath for 2 h, then brought to 1000 ml with distilled water and stored at 4° C. for later use.

4) Preparation of vitamin stock solution (100× concentrate):

| | |
|---|---|
| Nicotinic acid | 0.1 g |
| Vitamin B1 (Thiamine HCl) | 0.1 g |
| Vitamin B6 (Pyridoxine HCl) | 0.1 g |
| Glycine | 0.2 g |
| Inositol | 10 g |

Distilled water was added to dissolve the compounds and the resulting solution was brought to 1000 ml with distilled water and stored at 4° C. for later use.

5) Preparation of macroelement mother solution for MS basal medium (10× concentrate):

| | |
|---|---|
| Ammonium nitrate (NH₄NO₃) | 16.5 g |
| Potassium nitrate | 19.0 g |
| Potassium dihydrogen phosphate | 1.7 g |
| Magnesium sulplate | 3.7 g |
| Calcium chloride | 4.4 g |

These compounds were dissolved in distilled water and then the volume was brought to 1000 ml with distilled water at room temperature.

6) Preparation of microelement mother solution for MS basal medium (100× concentrate):

| | | |
|---|---|---|
| Potassium iodide | 0.083 | g |
| Boric acid | 0.62 | g |
| Magnesium sulplate | 0.86 | g |
| Sodium molybdate (Na₂MoO₄ • 2H₂O) | 0.025 | g |
| Copper sulphate (CuSO₄ • 5H₂O) | 0.0025 | g |

These compounds were dissolved in distilled water and then the volume was brought to 1000 ml with distilled water at room temperature.

7) 2,4-D stock solution, 6-BA stock solution, naphthaleneacetic acid (NAA) stock solution, indoleacetic acid (IAA) stock solution were all 1 mg/ml.

8) Glucose stock solution was 0.5 g/ml.

9) Preparation of AS stock solution: 0.392 g AS was weighed and dissolved in 10 ml DMSO.

### (3) Culture Media for Genetic Transformation of Rice

1) Induction Culture Medium:

| | | |
|---|---|---|
| N6max mother solution (10X) | 100 | ml |
| N6mix mother solution (100X) | 10 | ml |
| Fe²⁺ EDTA stock solution (100X) | 10 | ml |
| Vitamin stock solution (100X) | 10 | ml |
| 2,4-D stock solution | 2.5 | ml |
| Proline | 0.3 | g |
| CH | 0.6 | g |
| Sucrose | 30 | g |
| Phytagel | 3 | g |

Distilled water was added to a volume of 900 ml, and the pH value was adjusted to 5.9 with 1 N potassium hydroxide. The resulting mixture was boiled and brought to 1000 ml. The resulting medium was dispensed into 50 ml Erlenmeyer flasks (25 ml/flask), and the flasks were sealed and sterilized.

2) Callus Subculture Medium:

| | | |
|---|---|---|
| N6max mother solution (10X) | 100 | ml |
| N6mix mother solution (100X) | 10 | ml |
| Fe²⁺ EDTA stock solution (100X) | 10 | ml |
| Vitamin stock solution (100X) | 10 | ml |
| 2,4-D stock solution | 2.0 | ml |
| Proline | 0.5 | g/L |
| CH | 0.6 | g/L |
| Sucrose | 30 | g/L |
| Phytagel | 3 | g/L |

Distilled water was added to a volume of 900 ml, and the pH value was adjusted to 5.9 with 1 N potassium hydroxide. The resulting mixture was boiled and brought to 1000 ml. The resulting medium was dispensed into 50 ml Erlenmeyer flasks (25 ml/flask), and the flasks were sealed and sterilized

3) Pre-culture Medium:

| | | |
|---|---|---|
| N6max mother solution (10X) | 12.5 | ml |
| N6mix mother solution (100X) | 1.25 | ml |
| Fe²⁺ EDTA stock solution (100X) | 2.5 | ml |
| Vitamin stock solution (100X) | 2.5 | ml |
| 2,4-D stock solution | 0.75 | ml |
| CH | 0.15 | g/L |
| Sucrose | 5 | g/L |
| Agarose | 1.75 | g/L |

Distilled water was added to a volume of 250 ml, and the pH value was adjusted to 5.6 with 1 N potassium hydroxide. The resulting medium was sealed and sterilized. Prior to use, the medium was melted under heat and 5 ml glucose stock solution and 250 μl AS stock solution were added. The resulting medium was dispensed into Petri dishes (25 ml/dish).

4) Co-culture medium:

| | | |
|---|---|---|
| N6max mother solution (10X) | 12.5 | ml |
| N6mix mother solution (100X) | 1.25 | ml |
| Fe²⁺ EDTA stock solution (100X) | 2.5 | ml |
| Vitamin stock solution (100X) | 2.5 | ml |
| 2,4-D stock solution | 0.75 | ml |
| CH | 0.2 | g/L |
| Sucrose | 5 | g/L |
| Agarose | 1.75 | g/L |

Distilled water was added to a volume of 250 ml, and the pH value was adjusted to 5.6 with 1 N potassium hydroxide. The resulting medium was sealed and sterilized. Prior to use, the medium was melted under heat and 5 ml glucose stock solution and 250 μl AS stock solution were added. The resulting medium was dispensed into Petri dishes (25 ml/dish).

5) Suspension Medium:

| | | |
|---|---|---|
| N6max mother solution (10X) | 5 | ml |
| N6mix mother solution (100X) | 0.5 | ml |
| Fe²⁺ EDTA stock solution (100X) | 0.5 | ml |
| Vitamin stock solution (100X) | 1 | ml |
| 2,4-D stock solution | 0.2 | ml |
| CH | 0.08 | g/L |
| Sucrose | 2 | g/L |

Distilled water was added to a volume of 100 ml, and the pH value was adjusted to 5.4. The resulting medium was dispensed into two 100 ml Erlenmeyer flasks and the flasks were sealed and sterilized. Prior to use, 1 ml glucose stock solution and 100 μl AS stock solution were added.

6) Selective Medium:

| | | |
|---|---|---|
| N6max mother solution (10X) | 25 | ml |
| N6mix mother solution (100X) | 2.5 | ml |
| Fe²⁺ EDTA stock solution (100X) | 2.5 | ml |
| Vitamin stock solution (100X) | 2.5 | ml |
| 2,4-D stock solution | 0.625 | ml |
| CH | 0.15 | g/L |
| Sucrose | 7.5 | g/L |
| Agarose | 1.75 | g/L |

Distilled water was added to a volume of 250 ml, and the pH value was adjusted to 6.0. The resulting medium was sealed and sterilized. Prior to use, the medium was melted and 250 μl HN and 400 ppm CN were added. The resulting medium was dispensed into Petri dishes (25 ml/dish).

7) Pre-differentiation Medium:

| | | |
|---|---|---|
| N6max mother solution (10X) | 25 | ml |
| N6mix mother solution (100X) | 2.5 | ml |
| Fe²⁺ EDTA stock solution (100X) | 2.5 | ml |
| Vitamin stock solution (100X) | 2.5 | ml |
| 6-BA stock solution | 0.5 | ml |
| KT stock solution | 0.5 | ml |
| NAA stock solution | 50 | μl |
| IAA stock solution | 50 | μl |
| CH | 0.15 | g/L |
| Sucrose | 7.5 | g/L |
| Agarose | 1.75 | g/L |

Distilled water was added to a volume of 250 ml, and the pH value was adjusted to 5.9 with 1N potassium hydroxide. The resulting medium was sealed and sterilized. Prior to use, the medium was melted and 250 μl HN and 200 ppm CN were added. The resulting medium was dispensed into Petri dishes (25 ml/dish).

8) Differentiation Medium:

| | | |
|---|---|---|
| N6max mother solution (10X) | 100 | ml |
| N6mix mother solution (100X) | 10 | ml |
| Fe²⁺ EDTA stock solution (100X) | 10 | ml |
| Vitamin stock solution (100X) | 10 | ml |
| 6-BA stock solution | 2 | ml |
| KT stock solution | 2 | ml |
| NAA stock solution | 0.2 | ml |
| IAA stock solution | 0.2 | ml |
| CH | 1 | g/L |
| Sucrose | 30 | g/L |
| Phytagel | 3 | g/L |

Distilled water was added to a volume of 900 ml, and the pH value was adjusted to 6.0 with 1N potassium hydroxide. The resulting mixture was boiled and brought to 1000 ml. The resulting medium was dispensed into 50 ml Erlenmeyer flasks (50 ml/flask), and the flasks were sealed and sterilized.

9) Rooting Medium:

| | | |
|---|---|---|
| MSmac mother solution (10X) | 50 | ml |
| MSmic mother solution (100X) | 5 | ml |
| Fe²⁺ EDTA stock solution (100X) | 5 | ml |
| Vitamin stock solution (100X) | 5 | ml |
| Sucrose | 30 | g/L |
| Phytagel | 3 | g/L |

Distilled water was added to a volume of 900 ml, and the pH value was adjusted to 5.8 with 1N potassium hydroxide. The resulting mixture was boiled and brought to 1000 ml. The resulting medium was dispensed into the rooting tubes (25 ml/tube), and the tubes were sealed and sterilized.

### Example 3

### Determination of the Expression Level of Rice Endogenous OsNHAD Gene

RNA was extracted from ten various tissues collected at different growth stages of the rice variety “Zhenshan 97” (Oryza sativa L. ssp. Indica, a rice variety popularized in China) to determine the expression level of OsNHAD gene by real-time PCR. The ten tissues collected were: 1) callus; 2) seed; 3) three-day-old shoot; 4) leaf and root at trefoil stage; 5) flag leaf; 6) stem; 7) young spike shorter than 5 cm; 8) extruded spike; 9) glume; and 10) endosperm. Total RNA was extracted with TRIZOL reagent (Invitrogen Corp., performed according to the manufacturer's instruction), and reverse transcribed into cDNA with reverse transcriptase SSII (Invitrogen Corp., performed according to the manufacturer's instruction) under the following conditions: 65° C. for 5 min, 42° C. for 120 min, and 70° C. for 10 min. Using the above reverse transcribed cDNA as template, and using primers 5′-GCACAAAATCTCCCTCTATCCC T-3′ and 5′-CACCCTTTCATGACCCCG-3′, OsNHAD gene was specifically amplified by PCR (the amplification product was 73 bp in length). Meanwhile, primers AF: 5′-TGGCATCTCTCAGCACATTCC-3′ and AR: 5′-TGCACAAT GGATGGGTCAGA-3′ were used to specifically amplify rice Actinl gene (the amplification product is 76 bp in length) as internal control for quantitative analysis. The reaction conditions were: 95° C. for 5 min; and 40 cycles of 95° C. for 10 sec, 60° C. for 5 sec, and 72° C. for 34 sec. During the reaction the real-time fluorescence detection quantitative analysis was performed. Results showed that OsNHAD gene was expressed in all the tissues collected, with a relatively high level in leaves and endosperm (see FIG. 3).

The indica rice variety “Zhenshan 97” was used for expression profile analysis. The seeds were induced to germinate and cultivated under normal growth conditions for 18-20 days until the 4-leaf stage, when stress treatment and hormone treatment were carried out. Drought treatment was carried out by removing the young shoots from hydroponic solution and exposing them in air, with samples taken before treatment and 1, 3, 6, 12 and 24 hours after treatment. High salt treatment was carried out by transferring the young shoots from hydroponic solution to aqueous solution containing 200 mmol/L NaCl, with samples taken before treatment and when the leaves were slightly curled, half curled, completely curled, and rehydrated. Low temperature treatment was carried out by placing the young shoots into 4° C. artificial climate room, with samples taken before treatment and 1, 6, 12 and 24 hours after treatment. Hormone treatment was carried out by evenly spraying 100 μM abscisic acid (ABA) onto the surfaces of the rice plants, with samples taken before treatment and 30 minutes as well as 3, 6, 12 and 24 hours after treatment. All treatments and sampling were done under constant light. Total RNA was extracted from leaves using Trizol reagent (purchased from Invitrogen Corp.), transferred to membrane according to experimental procedures described (see Sambrook, J., E. F. Fritsch, and T. Maniatis, Molecular Cloning: a Laboratory Manual (3^{rd} edition), translated by Huang Peitang, Wang Jiaxi et al., Science Press (China), 2002 edition), and subjected to Northern hybridization using OsNHAD as the probe. Results showed that the expression of OsNHAD gene was strongly upregulated when induced by high salt, low temperature and ABA treatment and was slighted increased when induced by drought.

### Example 4

### Growth of T1 Transgenic Lines Overexpressing OsNHAD Gene Under Stress Conditions

In this example, five T1 lines overexpressing OsNHAD gene were selected for high salt stress experiment. The procedure was carried out as follows. Rice seeds from transgenic lines overexpressing OsNHAD gene were husked and disinfected (75% alcohol treatment for 3 minutes followed by 0.15% HgCl₂ treatment for 30 minutes, and rinsed with sterile water for several times), and then allowed to germinate on the ½ MS basal media containing 50 mg/L hygromycin. One day later, the rice seeds from wild-type control lines were placed on the ½ MS basal media without hygromycin and allowed to germinate. Two to three days later, well germinated and consistently grown seeds were transferred to the ½ MS basal media containing 0 mmol/L and 150 mmol/L NaCl, and cultivated in an illumination incubator (which simulated natural growth condition of 14-hour light and 10-hour darkness) for 10 days for phenotype observation and plant height determination. More than 30 plants were selected for each of the transgenic and wild-type plant lines, and each experiment was run in triplicate. The results obtained were as follows. Under normal conditions, the growth of the transgenic plants overexpressing OsNHAD gene cloned in the present invention was not distinctly different from control plants, while after stress treatment, the growth of the former was significantly better than the latter (P<0.01, t test). This indicated that the expression of OsNHAD gene could relieve the retarded growth of the plants caused by stress conditions, and increase the tolerance of transgenic rice plants to non-biological stresses (FIG. 5).

Although the expession of OsNHAD gene was also induced by low temperature and drought, the five T1 transgenic lines overexpressing OsNHAD gene in the present example did not show significant increase in tolerance to low temperature and drought. This is probably because the product that OsNHAD gene encodes is a Na⁺/H⁺ antiporter specifically involved in tolerance of the plant to high salt stress.

1. Use of a rice OsNHAD gene that enhances tolerance to salt stress in genetic improvement of salt tolerance of rice, wherein said gene is selected from one of the following nucleotide sequences:
1) the DNA sequence from positions 60 to 1649 of SEQ NO: 1 in the Sequence Listing; or
2) a DNA sequence that encodes the same protein as that encoded by 1).


SEQUENCE LISTING (2 sequences)

SEQ ID NO: 1 - DNA, 3252 bp, Oryza sativa
  gene (1)..(3252)
  CDS (60)..(1649)
ggcactctcactcacacggagagagagagagagagagactagactagtagtaccaattaatggcggcgctctcctcctgcttactcgccgccgtccgacctcatcctccacctccacctcgaccgctctccccttccttcatcccctctgccctccgccaccgccaccgcctcagccaagcgccgcccctcgccacctctctcccacgaccacgaccaccatggtgccgcttctccgcctcctcgccgccaccgccacccgacgaccctgacgactacgagctgttggacacgacaggaaattgtgatcctttatgctccgttgatgaagttagctcacagtattttgaagctaactacaagccaaaaaatgatcttcttaaagctttaactataatagcaacagctttggctggggcagctgcaataaaccattcctgggttgctgaacatcaggacattgcaatggtgctagtgtttgctctcggttatgcaggtatcatatttgaggaatcactagcgtttaacaaaagtggagttggattgctcatggccgtttgcctatgggttatcagaagtatcggggctccatctactgatgtagcagttcaagagttgagtcatacaaccgccgaagttagtgaaatagtctttttcttgctcggtgcaatgaccattgttgagattgttgatgcacatcaaggatttaagctagtgactgacaatatatctactcgaaatccaagaactcttctctgggtgatagggtttgtaactttcttcttgagttctatccttgacaatttgacttccactattgtgatggtttcattgcttcggaaactagtacctccatcagagtacagaaaattgttaggcgctgttgttgtgatatctgcaaatgctgggggtgcatggacaccaattggtgatgtgacgaccactatgttgtggattcatggtcagattacaacgttgaacacaatgcagggcttgtttcttccctcagttgtttcattggcagttccactggctctgatgtccctcacgagtgaagcaaatggatcttctcagaaatcttccagcttgctgtcatcagagcagatggctcctcgaggacaacttgtatttgctgttggccttggagccttagtgtttgttccagtgtttaaagctctcactgggctgccacctttcatgggcatgatgcttggtcttgcaactctttggatcctgacagatgcgatacattatggggactctggaaggcagagattgaaagttccacaagcgctgtcacggattgatacacaaggagttctatttttcttaggaattcttatgtcagttggcagcttggaatctgctggcattttgaggcagttggcgaactatctcgatgccaatattccgaatgccgaccttattgcaagcgctatcggtgtagcatcagcaattatagacaatgttccacttgttgctgcaacaatggggatgtgtggctttcatgggaatggaaaaggtggatttcttttggtatttccgcaaggtaaattgaacttttactctaataataagtggagctgtggaacaacttacacaatacagaatcgtatgttacagaacaagaaacttattcggtgtggttgatatttccctgtcgaactgttaaatccaatccatatatgctcatcatatctttcagtaattctgataagggctgggaacagggaaaaaaattgattttggactgcagcttggtttagcttctctataggaaaacaattgtgatatcatgcattgatggtacgtacgtaagggcaagattgtgcttttctgtaagggatgaaataccatgctttcattaattagttatagttttatgttttaaaccaagctcgcttatttgtttacggcataactttgtttatagatttgcacaagtgtcttctcaatgatttggatgtaaagtagattgcccacctttcttcaactatttgtgcttttcggagaattgcaacactaatttataagtccatcgtgctgttagtgtcatctcagaacctcaaatccttaacgattggtagtaagttgtgtgtacttgtacggaattagcaactacaagtgactaggcccgtagaatatatgtaaacattgaaatgatctatgtcttttgagtccacattgtatttataccgcttggttttgcaggtgagtggttttgcccttgcaggttatgcagctggtatcatcacttatctagctgcacaaaatctccctctatcccttcccacttcactagccgagatcccatttatctcggggtcatgaaagggtgaccagattcaggtatggaaatggacatattttattgtctggccagcttatgtaagcataatcaggatgtgttggcaaagaaaagatgtcccatgattacatgggatggaataaacgaaaaataaatatatgccctgatctccaacctccaagtttcccaacattgaccggaaggctttggggtgctggtgaactatggtatgctaagagaggagcgattttgataacacaagcttagaggctagagctgcacgcttgtaattcacgaattctattccatcttgttgagaagaaaagaggagctgcaccttgtgtaaaatttgaagatcctgaaatcatatcatcactgggtttatttttgccctgttgtttgcctacacttgttgggtacttgtgttttctgtactccgttcgaggaggtgaccattgctatcacactatgccattgaatcatttccttctaccttttgtgaagaggtgatatggttccatttgagtcaaagtcaagtcaaatgtaatggactaggaacatcatgtggggacagatgctttcccagaagcagaattgctacaccactactccctccgtcccaaaaaaaagacaaaccctggttttcgtgcccaatgtttgaccgtctgtcttatttaaaaaaattatgaaaaaaaataaaaagacaagttacgcataaaatattaattatgttttatcatctaacaacaataaaaatacaaattataaaaaaatttcatataagacggacagtcaaagttggatacgaaaacccagggtttgccttttttttaggacggagggagtagtgctgtgtctaccgggtctgcctgaagaaatgacaaacatggcctagggcattcctaatgcctattttggctgacaagggttacattgggaccatcc

SEQ ID NO: 2 - protein, 529 aa, Oryza sativa
MAALSSCLLAAVRPHPPPPPRPLSPSFIPSALRHRHRLSQAPPLATSLPRPRPPWCRFSASSPPPPPDDPDDYELLDTTGNCDPLCSVDEVSSQYFEANYKPKNDLLKALTIIATALAGAAAINHSWVAEHQDIAMVLVFALGYAGIIFEESLAFNKSGVGLLMAVCLWVIRSIGAPSTDVAVQELSHTTAEVSEIVFFLLGAMTIVEIVDAHQGFKLVTDNISTRNPRTLLWVIGFVTFFLSSILDNLTSTIVMVSLLRKLVPPSEYRKLLGAVVVISANAGGAWTPIGDVTTTMLWIHGQITTLNTMQGLFLPSVVSLAVPLALMSLTSEANGSSQKSSSLLSSEQMAPRGQLVFAVGLGALVFVPVFKALTGLPPFMGMMLGLATLWILTDAIHYGDSGRQRLKVPQALSRIDTQGVLFFLGILMSVGSLESAGILRQLANYLDANIPNADLIASAIGVASAIIDNVPLVAATMGMCGFHGNGKGGFLLVFPQGKLNFYSNNKWSCGTTYTIQNRMLQNKKLIRCG